(19) United States
(12) Patent Application Publication
(10) Pub. No.: US 2004/0266712 A1
(43) Pub. Date: Dec. 30, 2004

(54) Selective inhibition of vascular smooth muscle cell proliferation

(76) Inventors: Leslie M. McEvoy, Mountain View, CA (US); Michael J. Mann, Palo Alto, CA (US); Christi Parham, San Francisco, CA (US); Victor J. Dzau, Newton, MA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/819,500

(22) Filed: Apr. 6, 2004

Related U.S. Application Data
(60) Provisional application No. 60/461,626, filed on Apr. 8, 2003.

(51) Int. Cl. A61K 48/00

(52) U.S. Cl. 514/44

(57) ABSTRACT

The present invention concerns methods and means for selective inhibition of vascular smooth muscle cell (VMSC) proliferation, without negative impact on the proliferation of endothelial cells. In particular, the invention concerns the inhibition of VMSC proliferation without substantial inhibition of endothelial cell proliferation or function by delivery to a blood vessel in need of healing an E2F decoy oligonucleotide.

### CROSS-REFERENCE TO RELATED APPLICATIONS

This is a non-provisional application filed under 37 C.F.R. 1.53(b), claiming priority under U.S.C. § 119(e) to provisional Application Ser. No. 60/461,626, filed Apr. 8, 2003.

### BACKGROUND OF THE INVENTION

1. Field of the Invention

The present invention concerns methods and means for selective inhibition of vascular smooth muscle cell (VMSC) proliferation, without negative impact on the proliferation of endothelial cells. In particular, the invention concerns the inhibition of VMSC proliferation without substantial inhibition of endothelial cell proliferation or function by delivery to a blood vessel in need of healing an E2F decoy oligonucleotide.

2. Description of the Related Art

The E2F family of transcription factors plays a pivotal role in the control of cell cycle progression, and regulates the expression of numerous genes, including genes involved in cell cycle regulation, including those encoding c-Myc, c-Myb, Cdc2, proliferating-cell nuclear antigen (PCNA), Cyclin A, dihydrofolate reductase, thymidine kinase, and DNA polymerase α.

E2F is now recognized as a family of six heterodimeric complexes encoded by distinct genes, divided into two distinct groups: E2F proteins (E2F-1-E2F-6) and DP proteins (DP-1 and DP-2). The E2F proteins themselves can be divided into two functional groups, those that induce S-phase progression when over-expressed in quiescent cells (E2Fs 1-3), and those that do not (E2Fs 4-5). E2F-6 is functionally different in that its over-expression has been described to suppress the transactivational effects of co-expression of E2F-1 and DP-1. In addition, it has been reported that E2F-6 expression delays the exit from S-phase rather than inducing S-phase. The proteins from the E2F and DP groups heterodimerize to give rise the E2F activity. All possible combinations of E2F-DP complexes exist in vivo. Individual E2F-DP complexes invoke different transcriptional responses depending on the identity of the E2F moiety and the proteins that are associated with the complex. In addition homodimers of E2F molecules have also been described. (See, e.g. Zheng et al., Genes & Devel 13:666-674 (1999).)

Depending on whether they are associated with the retinoblastoma (Rb) family of pocket proteins, E2F proteins can act either as repressors or as activators of transcription (Hiebert et al. Genes & Devel 6:177-185 (1992); Weintraub et al., Nature 358:259-261 (2002)).

E2F transcription factors are responsible for activating a dozen or more genes that must be turned on during vascular cell growth and multiplication. Its blockade prevents the proliferation of these abnormal cells (neointimal hyperplasia) that eventually result in atherosclerotic lesions. As a result of their biological functions, E2F transcription factors have been implicated in neointimal hyperplasia, neoplasia glomerulonephritis, angiogenesis, and inflammation. Various members of the E2F family have also been described to play a role in cancer, and identified as targets for anti-cancer agents. For an overview of E2F family members, regulation and pathway see, e.g. Harbour, J. W., and Dean, D. C., Genes Dev 14, 2393-2409 (2000); Mundle, S. D., and Saberwal, G., Faseb J 17, 569-574 (2003); and Trimarchi, J. M., and Lees, J. A. Nat Rev Mol Cell Biol 3, 11-20 (2002).

E2F binding sites have been identified in the promoter regions of several cellular genes, and reported, for example, in the following publications: Farnham et al., Biochim. Biophys. Acta 1155:125-131 (1993); Nevins, J. R., Science 258:424-429 (1992); Shan et al., Mol. Cell. Biol. 14:299-309 (1994); Thalmeier et al., Genes Dev. 3:517-536 (1989); Delton et al., EMBO J. 11:1797-1804 (1992); Yamaguchi et al., Jpn. J. Cancer Res. 83:609-617 (1992).

Oligonucleotide decoys targeting E2F transcription factors have been described in PCT Publication No. WO 95/11687, published May 5, 1995, the entire disclosure of which is hereby expressly incorporated by reference.

Autologous vein remains the most widely used bypass conduit for the treatment of occlusive coronary and peripheral vascular disease, although failure rates in these grafts remain as high as 30% and 50% at 5 and 10 years, respectively (Angelini and Newby, Eur. Heart J. 10:273-280 (1989); Grondin, C. M., J. Thorac. Cardiovasc. Surg. 87:161-166 (1984)). VSMC proliferation and neointima formation provide wall thickening that relieves the increased wall stress brought on by exposure to the high-pressure arterial circulation. These activated VSMCs, along with dysfunctional endothelial cells, render the graft highly susceptible to accelerated atherosclerosis. (Faries et al., J. Vasc. Surg. 24:463-471 (1996); Hoch et al., Surgery 116-463-471 (1994); Zwolak et al., Arteriosclerosis 9:374-379 (1989)). Endothelial cell dysfunction, including reduction in endothelial cell nitric oxide synthase activity, decreased vasoreactivity, and increased expression of proinflammatory cell adhesion molecules, has been well documented in both experimental models of vein grafting and in human clinical specimens. (Cross et al., Ann. Surg. 208:631-638 (1988)). It has been demonstrated that inhibition of neointima formation by blockade of cell cycle regulatory gene expression leads to a significant improvement in endothelial cell function (Mann et al., J. Clin. Invest. 99:1295-1301 (1997)).

E2F oligonucleotide decoys are in clinical development as a means of altering the natural history of vein grafts, without the potential hazards of methods that require the introduction of oligonucleotides in vivo, and are expected to be of great clinical value in solving a vexing problem confronting all surgical repair of arteries in a variety of clinical circumstances. The U.S. Food and Drug Administration has granted Fast Track. designation for an E2F decoy molecule (Corgentech, Inc., South San Francisco, Calif.), which is designed to prevent blocking and failing of vein grafts used in coronary artery and peripheral arterial by-pass procedures.

Further representative references concerning E2F decoy therapy include: Morishita, R., G. H. Gibbons, M. Horiuchi, K. E. Ellison, M. Nakama, L. Zhang, Y. Kaneda, T. Ogihara, and V. J. Dzau. (1995). A gene therapy strategy using a transcription factor decoy of the E2F binding site inhibits smooth muscle proliferation in vivo. Proceedings of the National Academy of Sciences USA, 92, 5855-5859; Dzau, V. J., M. J. Mann, R. Morishita, and Y. Kaneda. (1996). Fusigenic viral liposome for gene therapy in cardiovascular diseases. Proceedings of the National Academy of Sciences USA, 93, 11421-11425; von der Leyen, H. E., M. J. Mann, and V. J. Dzau. (1996). Gene inhibition and gene augmentation for the treatment of vascular proliferative disorders. Semin Interv Cardiology, 1, 209-214; Kaneda, Y., R. Morishita, and V. J. Dzau. (1997). Prevention of restenosis by gene therapy. Annals of the NY Academy of Sciences, 811, 299-308, discussion 308-210; Mann, M. J., and V. J. Dzau. (1997). Genetic manipulation of vein grafts. Current Opinion in Cardiology, 12, 522-527; Mann, M. J., G. H. Gibbons, P. S. Tsao, H. E. von der Leyen, J. P. Cooke, R. Buitrago, R. Kernoff, and V. J. Dzau. (1997). Cell cycle inhibition preserves endothelial function in genetically engineered rabbit vein grafts. Journal of Clinical Investigation, 99, 1295-1301; Morishita, R., G. H. Gibbons, M. Horiuchi, M. Nakajima, K. E. Ellison, W. Lee, Y. Kaneda, T. Ogihara, and V. J. Dzau. (1997). Molecular Delivery System for Antisense Oligonucleotides: Enhanced Effectiveness of Antisense Oligonucleotides by HVJ-liposome Mediated Transfer. Journal of Cardiovascular Pharmacology, 2, 213-222; Braun-Dullaeus, R. C., M. J. Mann, and V. J. Dzau. (1998). Cell cycle progression: new therapeutic target for vascular proliferative disease. Circulation, 98, 82-89; Mann, M. J. (1998). E2F decoy oligonucleotide for genetic engineering of vascular bypass grafts. Antisense Nucleic Acid Drug Development, 8, 171-176; Morishita, R., G. H. Gibbons, M. Horiuchi, Y. Kaneda, T. Ogihara, and V. J. Dzau. (1998). Role of AP-1 complex in angiotensin II-mediated transforming growth factor-beta expression and growth of smooth muscle cells: using decoy approach against AP-1 binding site. Biochemistry and Biophysics Res Community, 243, 361-367; Poston, R. S., K. P. Tran, M. J. Mann, E. G. Hoyt, V. J. Dzau, and R. C. Robbins. (1998). Prevention of ischemically induced neointimal hyperplasia using ex-vivo antisense oligodeoxynucleotides. Journal of Heart and Lung Transplant, 17, 349-355; Tomita, N., M. Horiuchi, S. Tomita, G. H. Gibbons, J. Y. Kim, D. Baran, and V. J. Dzau. (1998). An oligonucleotide decoy for transcription factor E2F inhibits mesangial cell proliferation in vitro. American Journal of Physiology, 275, F278-284; Mann, M. J., G. H. Gibbons, H. Hutchinson, R. S. Poston, E. G. Hoyt, R. C. Robbins, and V. J. Dzau. (1999). Pressure-mediated oligonucleotide transfection of rat and human cardiovascular tissues. Proceedings of the National Academy of Sciences USA, 96, 6411-6416; Mann, M. J., A. D. Whittemore, M. C. Donaldson, M. Belkin, M. S. Conte, J. F. Polak, E. J. Orav, A. Ehsan, G. Dell'Acqua, and V. J. Dzau. (1999). Ex-vivo gene therapy of human vascular bypass grafts with E2F decoy: the PREVENT single-centre, randomised, controlled trial. Lancet, 354, 1493-1498; Poston, R. S., M. J. Mann, E. G. Hoyt, M. Ennen, V. J. Dzau, and R. C. Robbins. (1999). Antisense oligodeoxynucleotides prevent acute cardiac allograft rejection via a novel, nontoxic, highly efficient transfection method. Transplantation, 68, 825-832; Tomita, S., N. Tomita, T. Yamada, L. Zhang, Y. Kaneda, R. Morishita, T. Ogihara, V. J. Dzau, and M. Horiuchi. (1999). Transcription factor decoy to study the molecular mechanism of negative regulation of renin gene expression in the liver in vivo. Circulation Research, 84, 1059-1066; von der Leyen, H. E., R. Braun-Dullaeus, M. J. Mann, L. Zhang, J. Niebauer, and V. J. Dzau. (1999). A pressure-mediated nonviral method for efficient arterial gene and oligonucleotide transfer. Human Gene Therapy, 10, 2355-2364; Ehsan, A., and M. J. Mann. (2000). Antisense and gene therapy to prevent restenosis. Vascular Medicine, 5, 103-114; Mann, M. J. (2000). Gene therapy for vein grafts. Current Cardiology Reports, 2, 29-33; Mann, M. J. (2000). Gene therapy for peripheral arterial disease. Molecular Medicine Today, 6, 285-291; Mann, M. J., and V. J. Dzau. (2000). Therapeutic applications of transcription factor decoy oligonucleotides. Journal of Clinical Investigation, 106, 1071-1075; Tomita, N., R. Morishita, S. Tomita, G. H. Gibbons, L. Zhang, M. Horiuchi, Y. Kaneda, J. Kaneda, J. Higaki, T. Ogihara, and V. J. Dzau. (2000). Transcription factor decoy for NFkappaB inhibits TNF-alpha-induced cytokine and adhesion molecule expression in vivo. Gene Therapy, 7, 1326-1332; Ehsan, A., M. J. Mann, G. Dell'Acqua, and V. J. Dzau. (2001). Long-term stabilization of vein graft wall architecture and prolonged resistance to experimental atherosclerosis after E2F decoy oligonucleotide gene therapy. Journal of Thoracic Cardiovascular Surgery, 121, 714-722. The complete disclosures of the cited references are hereby expressly incorporated by reference.

Whereas the endothelium of genetically engineered vein grafts may be spared chronic activation by the paracrine influences of the underlying neointimal cells, acute endothelial healing remains an important component of the response to the injury associated with vein graft harvest and implantation. Little is known, however, about the acute healing response of the endothelial cell monolayer to the grafting procedure or the effects of cell cycle inhibitory therapy on that response.

There is a clinical need for methods that are capable of blocking cell cycle progression in VSMC, without hindering endothelial cell proliferation in the same vessel. In particular, there is a clinical need for treatment modalities that can prevent smooth muscle cell proliferation, without limiting normal endothelial healing following traumatic or biological injury to the vascular endothelium.

### SUMMARY OF THE INVENTION

The invention concerns a method for selective inhibition of vascular smooth muscle cell proliferation comprising: (a) delivering to a blood vessel in need of endothelial healing an E2F decoy oligonucleotide, and (b) determining that vascular smooth muscle cell proliferation is inhibited without substantial inhibition of endothelial cell proliferation or function.

The blood vessel can, for example, be a vein, a vein graft or an artery.

The E2F oligonucleotide decoy (ODN) can be delivered by any method known in the art including, for example, pressure-mediated transfer. Delivery can be ex vivo, in vitro or in vivo.

In a particular embodiment, step (b) of the above method comprises monitoring endothelial healing following delivery of the E2F decoy oligonucleotide.

In another embodiment, the E2F decoy oligonucleotide is delivered to a blood vessel of a mammal following vascular trauma, such as trauma due to a surgical procedure, vein or artery grafting or angioplasty. The mammal preferably is a human.

In another embodiment, the E2F decoy oligonucleotide is delivered within one week following vein or artery grafting or angioplasty.

In a further embodiment, the E2F decoy oligonucleotide is delivered during or immediately following vein or artery grafting or angioplasty.

In a still further embodiment, the E2F decoy oligonucleotide is delivered by coating or impregnating an implantable device, where the implantable device can, for example, be a cardiac stent, a renal stent, e.g. a renal artery stent, or an artificial conduit.

In a different aspect, the invention concerns an implantable device adapted for delivery of a biologically active agent to the site of a traumatized blood vessel comprising an effective amount of an E2F decoy oligonucleotide.

In a further aspect, the invention concerns a kit comprising an implantable device adapted for delivery of a biologically active molecule to the site of a traumatized blood vessel and a unit dosage form comprising an E2F decoy oligonucleotide.

In a particular embodiment, the unit dosage form is labeled for use in treating or inhibiting stenosis or restenosis.

In yet another aspect, the invention concerns a method for prevention of stenosis or restenosis, comprising (a) delivering to a blood vessel at risk of stenosis or restenosis an E2F decoy oligonucleotide, and (b) determining that vascular smooth muscle cell proliferation is inhibited without substantial inhibition of endothelial cell proliferation or function.

In a different aspect, the invention concerns a method for the treatment of stenosis or restenosis, comprising (a) delivering to a blood vessel exhibiting signs of stenosis or restenosis an E2F oligonucleotide, and (b) determining that vascular smooth muscle cell proliferation is inhibited without substantial inhibition of endothelial cell proliferation or function.

### BRIEF DESCRIPTION OF THE DRAWINGS

FIG. 1 shows scanning electron photomicrographs of jugular vein, carotid artery, and vein graft endothelial cells (magnification×1000). A. Ungrafted jugular vein; B. Contralateral carotid artery; C. Vein graft, postoperative day (POD) 1; D. Vein graft, POD 3; E. Vein graft, POD 7. Time course of change in endothelial cell density (F) and number (G) over 14 days (*P<0.001 vs POD 1) (n=4 for each group).

FIG. 2. BrdU labeling of endothelial cells. Light microscopy photomicrographs of Hautchen preparations for A. control jugular vein, and B. postoperative day (POD) 2 vein graft (magnification×400). C. Time course of endothelial proliferative response (*P<0.001 vs POD 1) (n=3 for each group).

FIG. 3. Fluorescent micrographs of rabbit jugular vein segments transfected with FITC-labeled ODN. A. Blue fluorescent staining with Hoechst 33342 identifying vessel wall nuclei. B. Nuclear localization of labeled ODN throughout vessel wall (magnification×400), including endothelial cells on luminal surface (arrow). C. Representative Western blot demonstrating low levels of PCNA protein expression in ungrafted jugular vein (lane 1) and in E2F decoy-treated grafts (lane 3) compared with untreated (lane 2) and control ODN-treated (lane 4) grafts.

FIG. 4. Endothelial cell response to E2F decoy ODN treatment. A. Scanning electron microscopy; B. Hautchen BrdU labeling index (n=3 for each group). POD indicates postoperative day.

FIG. 5. Nuclear localization of ODN. Light microscopy phoromicrograph of Hautchen preparation demonstrating nuclear localization of biotinylated ODN in vein graft endothelial cells at time of grafting (magnification×400).

FIG. 6. Electrophoretic mobility shift assay with human aortic smooth muscle cells.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

The terms “oligonucleotide decoy,” “oligodeoxynucleotide decoy,” and “ODN” are used interchangeably and in the broadest sense, and refer to short, linear and/or circular, double and/or single stranded DNA and/or RNA molcules, which bind to and interfere with a biological function of a transcription factor, such as E2F.

The term “E2F” is used herein in the broadest sense and includes all naturally occurring E2F molecules of any animal species, including E2F-1, E2F-2, E2F-3, E2F-4, E2F-5, and E2F.

The term “transcription factor binding sequence” is a short nucleotide sequence to which a transcription factor binds. The term specifically includes naturally occurring binding sequences typically found in the regulatory regions of genes the transcription of which is regulated by one or more transcription factors. The term further includes artificial (synthetic) sequences, which do not occur in nature but are capable of competitively inhibiting the binding of the transcription factor to a binding site in an endogenous gene.

The term “implantable device” is used in the broadest sense, and refers to any device that is capable or retaining and releasing a biologically active agent at the site of a traumatized blood vessel, either in vivo or ex vivo. Thus, the implantable device includes, without limitation, devices that can be placed into the lumen of a blood vessel, such as stents or catheters, or on the exterior of a blood vessel, such as a mesh, covering or wrap, as well as devices which become part of the blood vessel, such as natural or synthetic grafts, or artificial conduits. The implantable device may contain the biologically active agent, e.g. an E2F decoy oligonucleotide in the form of a composition, which might include a solid or liquid carrier, or matrix, such as a paste, gel, or permeable membrane. By way of example, and without limitation, the implantable device may be an artificial conduit, a stent, or a catheter. The blood vessels include, without limitation, coronary, renal, femoral, carotid, and peripheral vessels.

The term “blood vessel in need of endothelial healing” is used in the broadest sense, and refers to a blood vessel subject to any vascular trauma or injury that requires endothelial cell proliferation for repair. Included within the definition, are vascular traumas due to organ transplantation, such as heart, kidney, liver transplants, vascular surgery, angioplasty, e.g. balloon angioplasty, vascular graft procedures, placement of mechanical shunt, e.g. hemodialysis shunt used for arteriovenous communications, and placement of intravascular stent, including metallic, plastic and biodegradable polymer stents.

The term “proliferation” is used to refer to an increase in cell number, such as by mitosis.

The term “vascular smooth muscle cell” is used to refer to any type of vascular smooth muscle cells but preferably excludes neoplastic vascular smooth muscle cells (cancer cells).

The term “without substantial inhibition of endothelial cell proliferation or function” is used to describe the absence of an inhibitory process that would result in a detectable absence, inhibition, or slowing down of the healing of a blood vessel in need of endothelial healing, such as a traumatized blood vessel.

The term “mammal” as used herein refers to any animal classified as a mammal, including humans, higher primates, cows, horses, dogs and cats. In a preferred embodiment of the invention, the mammal is a human.

B. Detailed Description

The present invention concerns methods for selective inhibition of vascular smooth muscle cell proliferation by delivery to a blood vessel in need of endothelial healing an E2F decoy oligonucleotide (ODN).

E2F Oligonucleotide Decoy Molecules

The use of decoy ODNs for the therapeutic manipulation of gene expression was first described in PCT Publication No. WO 95/11687, published May 4, 1995, the entire disclosure of which is hereby expressly incorporated by reference, and in related scientific publications. Thus, Morishita et al. reported the treatment of rat carotid arteries at the time of balloon injury with ODNs bearing the consensus binding site for the E2F family of transcription factors (Proc. Natl. Acad. Sci. USA 92:5855-5859 (1995). It was found that a decoy specific to the E2F-1 isoform blocked smooth muscle proliferation and neointimal hyperplasia in injured vessels.

E2F oligonucleotide decoys (ODNs) are known in the art and described in the background art and examples of the present specification. Although short double-stranded DNA oligomers are most frequently used as transcription factor ODNs, structural modifications intended to enhance the stability and/or affinity or biological activity of these molecules are also known. For example, two strands of DNA can be cross-linked to for a single-stranded molecule folded on itself, either via photo-crosslinking (Iwase et al., Mucleic Acids Symp. Ser. 1997-203-204) or by the introduction of covalently linked, non-nucleotide bridge (Amoah-Apraku et al., Kidney Int. 57:83-91 (2000)). RNA decoy ODNs have been described that bind transcription factors via a aptameric interaction, as opposed to the naturally occurring sequence-directed binding site interaction (Iwase et al., supra). Circular decoy molecules assuming a dubbell configuration (Lebruska and Mather, Biochemistry 38:3168-3174 (1999)), and single-stranded decoys with a palindromic sequence that can fold on themselves (Hosoya et al., FEBS Lett. 461:136-140 (1999)) have also been described. For further details see, e.g. Mann and Dzau, J, Clin. Invest. 106:1071-1075 (2000).

Synthetic nucleotides may be modified in a variety of ways, see, e.g. Bielinska et al. Science 25); 997 (1990). Thus, oxygens may be substituted with nitrogen, sulfur or carbon; phosphorys substituted with carbon; deoxyribose substituted with other sugars, or individual bases substituted with an unnatural base. In each case, any change will be evaluated as to the effect of the modification on the binding ability and affinity of the oligonucleotide decoy to the E2F trascription factor, effect on melting temperature and in vivo stability, as well as any deleterious physiological effects. Such modifications are well known in the art and have found wide application for anti-sense oligonucleotide, therefore, their safety and retention of binding affinity are well established (see, e.g. Wagner et al. Science 260:1510-1513 (1993)).

Examples of modified nucleotides, without limitation, are: 4-acetylcytidin, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β,D-galactosylqueuosine, 2′-O-methylguanosine, inosine, N6-isopentenyladenosine 1-metyladenosine, 1-methylpseudouridine, 1-methylguanosine, 1-methylinosine, 2,2-dimethylguanosine, 2-methyladenosine, 2-methylguanosine 3-methylcytidine 5-methylcytidine, N6-methyladenosine, 7-methylguanosine, 5-methylaminomethyl-2-thiouridine, β, D-mannosylqueosine, 5-methoxycarbonylmethyl-2-thiouridine, 5-metoxycarbonalmethyluridine, 5-methoxyuridine, 2-methylthio-N6-isopentenyladenosine, N-((9-beta-D-ribofuransyl-2-methylthiopurine-6-yl)carbamoyl)threonine, N-((9-beta-D-ribofuranosylpurine-6-yl)N-methylcarbamoyl)threonine, uridine-5-oxyacetic acid-methylester uridine-5-oxyacetic acid, wybutoxosine, pseudouridine queuosine, 2-thiocytidine, 5-methyl-2-thiouridine, 2-thiouridine, 4-thiouridine, 5-methyluridine, N-((9-beta-D-ribofuransylpurine-6-yl)-carbamoylthreonine, 2′-O-methyl-5-methyluridine, 2′-O-methyluridine, 3-(3-3-amino-3-carboxy-propyl)uridine(acp3)u, and wybutosine.

In addition, the nucleotides can be linked to each other, for example, by a phosphoramidate linkage. This linkage is an analog of the natural phosphodiester linkage such that a bridging oxygen (—O—) is replaced with an amino group (—NR—), wherein R typically is hydrogen or a lower alkyl group, such as, for example, methyl or ethyl.

The E2F decoy molecules of the present invention can be synthesized by standard phosphodiester or phosphoramidate chemistry, using commercially available automatic synthesizers.

Therapeutic Targets

In one aspect, the present invention concerns method useful in the treatment of vascular trauma of any reason, including any physical or biological injury, such as, for example injury due to a surgical procedure, such as vein or artery grafting or angioplasty. Thus, a specific clinical target for using the methods of the present invention is the prevention and treatment of neointimal hyperplasia, the pathological process that underlies graft atherosclerosis, stenosis, and the majority of vascular graft occlusions. Neointimal hyperplasia is commonly seen after various forms of vascular injury, and is a major component of the vein graft's response to harvest and surgical implantation into high-pressure arterial circulation.

The methods of the present invention find further utility in the prevention and treatment of vascular injuries and vascular proliferative diseases or conditions characterized by undesired vascular smooth muscle cell proliferation, where it is important to maintain the integrity of the vascular endothelium.

In particular, the methods of the present invention find clinical use in the prevention and treatment of coronary heart disease, the single leading killer of American men and women, that caused over 450,000 deaths in the United States in 1998, according to the American Heart Association.

In addition, the methods of the present invention are useful in the prevention and treatment of peripheral vascular disease, which is characterized by atherosclerotic narrowing of peripheral arteries and, as a result, adversely affects blood circulation. In early clinical stages, the disease manifests itself in leg pain, but if left untreated, it can develop into gangrene, necessitating amputation of the limb, and substantial and irreversible morbidity and mortality.

Administration of the E2F Decoys

A preferred mode of delivering the E2F decoys of the present invention is pressure-mediated transfection, as described, for example, in U.S. Pat. Nos. 5,922,687 and 6,395,550, the entire disclosures of which are hereby expressly incorporated by reference. In brief, the E2F decoy molecules are delivered to cells in a tissue by placing the decoy nucleic acid in an extracellular environment of the cells, and establishing an incubation pressure around the cells and the extracellular environment. The establishment of the incubation pressure facilitates the uptake of the nucleic acid by the cells, and enhances localization to the cell nuclei.

More specifically, a sealed enclosure containing the tissue and the extracellular environment is defined, and the incubation pressure is established within the sealed enclosure. In a preferred embodiment, the boundary of the enclosure is defined substantially by an enclosing means, so that target tissue (tissue comprising the target cell) is subjected to isotropic pressure, and does not distend or experience trauma. In another embodiment, part of the enclosure boundary is defined by a tissue. A protective means such as an inelastic sheath is then placed around the tissue to prevent distension and trauma in the tissue. While the incubation pressure depends on the application, incubation pressures about 300 mmHg-1500 mmHg above atmospheric pressure, or at least about 100 mmHg above atmospheric pressure are generally suitable for many applications.

The incubation period necessary for achieving maximal transfection efficiency depends on parameters such as the incubation pressure and the target tissue type. For some tissue, such as human vein tissue, an incubation period on the order of minutes (>1 minute) at low pressure (about 0.5 atm) is sufficient for achieving a transfection efficiency of 80-90%. For other tissue, such as rat aorta tissue, an incubation period on the order of hours (>1 hour) at high pressure (about 2 atm) is necessary for achieving a transfection efficiency of 80-90%.

Suitable mammalian target tissue for this type of delivery includes blood vessel tissue (in particular veins used as grafts in arteries), heart, bone marrow, and connective tissue, liver, genital-urinary system, bones, muscles, gastrointestinal organs, and endocrine and exocrine organs. A method of the present invention can be applied to parts of an organ, to a whole organ (e.g. heart), or to a whole organism. In one embodiment a nucleic acid solution can be perfused into a target region (e.g. a kidney) of a patient, and the patient is subject to pressure in a pressurization chamber. Specific applications include the treatment of allografts (grafts derived from a different subject than the transplant patient) and syngrafts (grafts derived from the transplant patient).

For other applications, the E2F decoys of the present invention can be administered by other conventional techniques. For example, retrovial transfection, transfection in the form of liposomes are among the known methods suitable for transfection. For details see also Dzau et al., Trends in Biotech 11:205-210 (1993); or Morishita et al., Proc. Natl. Acad. Sci. USA 90:8474-8478 (1993). Where administered in liposomes, the decoy concentration in the lumen will generally be in the range of about 0.1 μM to about 50 μM per decoy, more usually about 1 μM to about 10 μM, most usually about 3 μM.

For other techniques, the most suitable concentration can be determined empirically. The determination of the appropriate concentrations and doses is well within the competence of one skilled in the art. Optimal treatment parameters will vary depending on the indication, decoy, clinical status of the patient, etc., and can be determined empirically based on the instructions provided herein and general knowledge in the art.

The decoys may be administered as compositions comprising individual decoys or mixtures of decoys. Usually, a mixture contains up to 6, more usually up to 4, more usually up to 2 decoy molecules.

In certain embodiments, the E2F decoy oligonucleotide is delivered to a blood vessel of a mammal following vascular trauma, such as trauma due to a surgical procedure, vein or artery grafting or angioplasty. Delivery can be performed during or following angioplasty, preferably within a short period of time after angioplasty. For such applications, the E2F decoy may, for example, be delivered by coating or impregnating an implantable device, where the implantable device can, for example, be a cardiac stent, a renal stent, e.g. a renal artery stent, or an artificial conduit.

Cardiac stents are implanted during procedures, such as angioplasty, to help keep open arteries that supply the heart with oxygen-rich blood. Coronary angioplasty is a minimally invasive procedure to open arteries that are blocked or narrowed by cholesterol and other fatty deposits. During angioplasty, typically a catheter is inserted into the clogged artery, usually through the groin/upper thigh artery, and threaded up to the heart. After the clogged artery is cleared may be permanently implanted. A stent is a scaffold-like tube made of wire mesh that adheres to the walls of the artery to keep it propped open allowing blood to flow freely. Stenting helps reduce the risk of restenosis or “re-narrowing” of the artery.

http://www.aurorahealthcare.org/services/cardiac/treatments/—ERS Coated stents are treated with a substance that makes it difficult for scar tissue or cholesterol to collect at the stent site. Thus, in the methods of the present invention, the stent may be pre-coated with an E2F ODN and optionally with one or more further substances, such as, for example, heparin, antibiotics, drugs that suppress the immune system, etc. Coated stent are delivered the same way as uncoated stents.

Catheters suitable for use in conjunction with the methods of the present invention are described, for example, in PCT publication No. WO 00/20066 published Apr. 13, 2000, but the methods of the invention can be performed also using other commercially available catheters.

Further details of the invention will be apparent from the following non-limiting Examples.

### EXAMPLE 1

Methods

Oligonucleotides

The double-stranded E2F decoy phosphorothioate E2F deoy oligonuclotide (ODN) was custom synthesized (Keystone-Biosource) with the sequence 5′-CTAGATTTCCCGCG-3′ (SEQ ID NO: 1) annealed to 5′-GATCCGCGGGAAAT-3′ (SEQ ID NO: 2), containing the E2F consensus binding sequence of the human c-myc promoter. The scrambled ODN, used as a control, has the following sequence: 5′-TCCAGCTTCGTAGC-3′ (SEQ ID NO: 3) annealed to 5′-GCTAGCTACGAAGC-3′ (SEQ ID NO: 4). ODN was dissolved at a concentration of 40 μmol/L in normal saline solution. Scrambled ODN labeled with either fluorescein-isothicyanate (FITC) or biotin at the 3′-end of one strand was used for fluorescent or light microscopic analysis of ODN distribution after transfection, respectively.

Vein Graft Model and Ex Vivo Transfection

Jugular vein-to-carotid artery interposition grafting in New Zealand White rabbits (weight, 3 to 3.5 kg) was performed as previously described (Ehsan et al., J. Thorac Cardiovasc. Surg. 121:714-722 (2001)). Briefly, a “no-touch” technique was used to harvest a 2.5-cm segment of vein, which was either left in normal saline at ambient pressure or treated with pressure-mediated delivery of ODN for 10 minutes at a nondistending pressure of 300 mm Hg. (Ehsan, et al. Long-term stabilization of vein graft wall architecture and prolonged resistance to experimental atherosclerosis after E2F decoy oligonucleotide gene therapy. J Thorac Cardiovasc. Surg. 121:714-722 (2001); Mann, et al., Pressure-mediated oligonucleotide transfection of rat and human cardiovascular tissues. Proc. Natl. Acad. Sci., USA, 96:6411-6416 (1999)). The vein graft was then anastomosed with 7-0 polypropylene sutures. The Harvard Medical Area Standing Committee on Animals approved the use of animals, and all animal care complied with the “Guide for the Care and Use of Laboratory Animals” (NIH Publication No. 80-23, revised 1985).

Häutchen Preparation

En face endothelial preparations of pressure-fixed vessels (100 mm Hg with 10% formalin for 10 minutes) were prepared with bromodeoxyuridine (BrdU) immunohistochemical staining (Zymed), as previously described. (Schwartz et al., Lab Invest. 28:699-707 (1983)). Briefly, flattened specimens were dehydrated and glued endothelial surface down onto 10% parlodion-coated glass slides. The vessel wall and subendothelium were peeled away, and the endothelial cells were applied to 5% gelatin-coated (Difco Laboratories) glass slides. The parlodion was dissolved, and slides were rehydrated for light microscopy. At time points beyond 1 to 2 days, Häutchen preparations failed to yield an endothelial monolayer because of the adherence of neointimal cells to the parlodion glue.

Silver Staining and Scanning Electron Microscopy

Vessels were harvested at 1, 3, 7, and 14 days after operation and pressure fixed. Sliver stained (0.3% silver nitrate over 20 seconds), opened, and dehydrated samples underwent critical point drying with liquid CO₂ and sputter coating with a gold-palladium alloy. The preparation was examined with the use of an Amray 1000 A scanning electron microscope. Silver staining of the endothelial cell borders allowed discrimination of endothelial cells from any adherent inflammatory cells, and determination of endothelial cell density was made on 8 sections of each specimen.

BrdU Incorporation and Immunohistochemistry

BrdU was administered at 18 hours (100 mg/kg SC and 30 mg/kg IV) and 12 hours (30 mg/kg IV) before harvest. BrdU labeling (Zymed) was carried out on either 5-μm frozen sections or with the use of the Häutchen preparation. BrdU-labeled and total nuclei were counted in 8 fields per sample at 400×.

Western Blot Analysis

Whole graft lysates were separated in a 10% SDS-polyacrylamide gel and transferred to a nitrocellulose membrane (Hybond ECL, Amersham Life Sciences). Protein concentrations were determined by the Bradford method (Biorad). The membrane was incubated with a polyclonal antibody for proliferating cell nuclear antigen (PCNA) (Santa Cruz Biotechnology Inc) for 1 hour and developed by the ECL system (Amersham Life Sciences). Signal intensities were quantified (NIH Image 1.52), and the results were expressed in arbitrary units per microgram of protein.

Endothelial and Smooth Muscle Cell Cultures, ODN Treatment, and Growth Assays

Human umbilical vein endothelial cells (HUVEC) and umbilical artery smooth muscle cells (HUASMC) were grown according to the supplier's recommendations (Clonetics Corporation). Confluent cells were synchronized in basal medium for 48 hours. The cells were restimulated with growth media for 24 hours and pulse-labeled with [3H]thymidine for 4 hours. ^{[3H]}thymidine incorporation (cpm) was measured in cell lysates as described. (Braun-Dullaeus R. C., et al. A novel role for the cyclin-dependent kinase inhibitor p27 (Kip 1) in angiotension II-stimulated vascular smooth muscle cell hypertrophy. J Clin Invest. 104:815-823 (1999). Where indicated, ODN (5 μmol/L) were added to the culture 24 hours before serum stimulation. Transfection efficiency was assessed by fluorescent microscopy of cells transfected with FITC-labeled ODN.

Electrophoretic Mobility Shift Assay

Nuclear extracts were prepared as previously described. (Horiuchi et al., J. Biol. Chem. 266:16247-16254 (1991); Horiuchi et al., J. Clin. Invest. 92:1805-1811 (1993)). Under similar conditions, the isolation of nuclear extract has not disrupted the sequence specific binding of phosphorothioate oligonucleotides to nuclear protein. (Park, Y. G., et al. Dual blockade of cyclic AMP response element—(CRE) and AP-1-directed transcription by CRE-transcription factor decoy oligonucleotide: gene-specific inhibition of tumor growth. J. Biol. Chem. 274:1573-1580 (1999). A peak of E2F binding activity was observed at 6 hours in preliminary experiments. Purified monoclonal E2F-1 antibody (PharMingen) was used in supershift assays as previously described (Horiuchi et al., 1991, supra, Horiuchi et al., 1993, supra).

Statistical Analysis

All results are expressed as mean+/−SEM. One-way ANOVA was used to compare differences between groups. A value of P<0.05 was considered to indicate a statistically significant difference, with Bonferroni correction for multiple comparisons.

Results

Endothelial Cell Healing

Vein graft endothelial barrier function was assessed macroscopically at days 1, 3, and 7 after grafting, with Evans blue dye administered intravenously (20 mg/kg in normal saline solution) 10 minutes before graft harvest, and was compared with ungrafted vein and carotid artery. At day 1, blue staining was present only at the anastamoses and was absent at days 3 and 7, whereas the body of the graft was devoid of staining at all time points (data not shown). The interposition of a vein into an arterial vessel leads to an acute distention of the vein graft and consequently a significant increase in surface area. Harvested vein segments, all 2.5 cm in length, were perfusion-fixed, opened lengthwise, and laid flat for measurement of surface area. A statistically significant change in the surface area of the graft compared with the ungrafted vein was observed at day 1 (291±19 mm² versus 183±16 mm²2, respectively, P<0.05). Further increase in graft surface area occurred up to day 7, at which time a maximum area of 361±39 mm² was reached (n=4 for each time point).

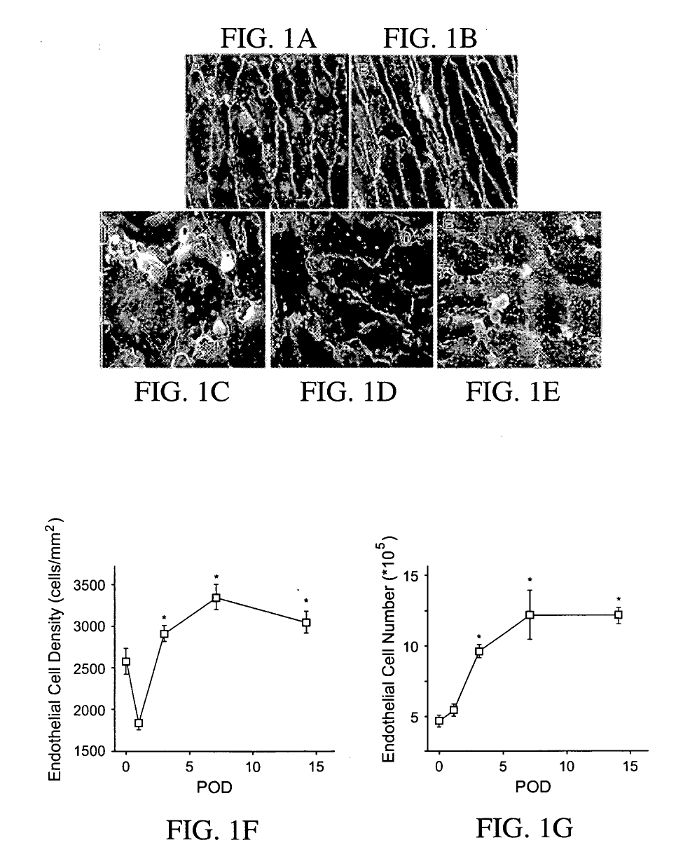

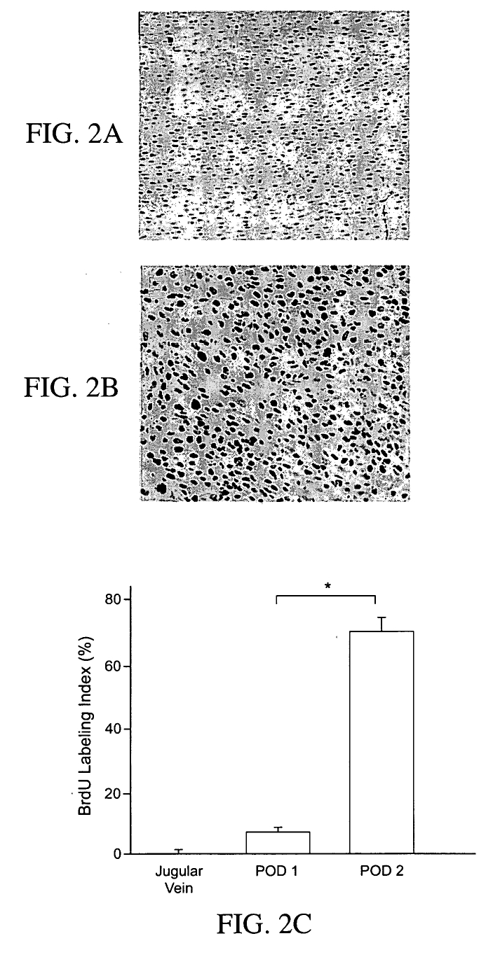

Scanning electron microscopy was performed on the endothelial surface after silver staining at 1, 3, 7, and 14 days after implantation and was compared with ungrafted vein and carotid artery (n=4 for all groups). At day 1, the grafts were noted to have endothelial cell loss only in the first 1 to 2 mm beyond the anastamosis, whereas the remainder of the graft showed no areas of denudation. Because of the acute increase in surface area, the cell densities of the day-1 grafts were significantly decreased (1819±61 cells/mm2) compared with those of the ungrafted veins (2587±140 cells/mm²) (P<0.05). Cell density at day 3 was 2897±103 cells/mm² (P<0.05 when compared with day 1). Endothelial cell density plateaued at 3338±157 cells/mm² at day 7 at a level slightly below that seen in the contralateral carotid artery (3409±133 cells/mm²). The number of endothelial cells present on the graft at each time point could be calculated by multiplication of the cell density by the graft surface area. The ungrafted vein was found to have 4.7×10⁵±2.9×10⁴ cells per 2.5-cm graft, whereas day-1 grafts had 5.3×10⁵±2.7×10⁴ cells (P<0.05). However, by day 3, the number of cells had increased to 9.6×10⁵±3.9×10⁴, and to 1.2×10⁶±1.7×10⁵ at day 7 and 1.2×10⁶±5.6×10⁴ at day 14 (P<0.05 when compared to day 1 and the ungrafted vein). These data collectively suggest that a burst of endothelial proliferation occurs in the vein graft between postoperative days 1 and 3 and that an equilibrium of cell number is reached by day 7 (FIG. 1). To further document this endothelial proliferative response, en face immunohistochemical staining of endothelial cells labeled with BrdU was performed by means of the Häutchen technique (n=3 for all time points). A labeling index of <1% was noted in the ungrafted vein, whereas an index of 6.6±4.3% was observed 1 day after grafting. At day 2, however, the BrdU labeling index had dramatically increased to 71.8±2.7% (FIG. 2). Tissue from animals not treated with BrdU and sections of BrdU-treated tissue stained with nonspecific IgG isotype antibody served as negative controls.

Effect of E2F Decoy ODN Treatment on Graft Endothelial Cell and VSMC Proliferative Response

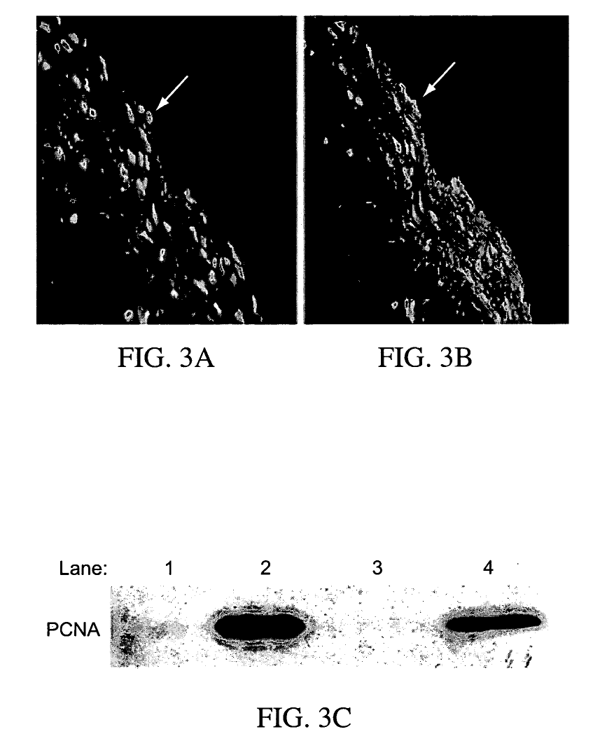

Using a previously described, ex vivo, nondistending, pressure-mediated delivery of ODN to the vein graft wall (Mann et al., Proc. Natl. Acad. Sci. USA 96:6411-6416 (1999)), as a first step, efficient uptake of FITC-labeled ODN in both endothelial cells and VSMC was demonstrated (FIG. 3, A and B). The effective delivery of functional E2F decoy ODN was further confirmed by Western blot analysis for PCNA in whole vascular graft 4 days after transfection. Our findings demonstrated that E2F decoy ODN treatment yielded a sequence specific inhibition of PCNA upregulation in the vessel wall that is predominantly composed of VSMC (FIG. 3C and Table). Grafts harvested 7 days after implantation, a time when VSMC proliferation is known to be at its peak, underwent assessment of BrdU incorporation in medial cells on 5-μm cross sections. The findings confirmed that E2F decoy ODN treatment inhibited medial VSMC proliferation. Grafts treated with the E2F decoy ODN had a significantly lower labeling index compared with either vehicle or scrambled ODN-treated grafts, as shown in the Table below.

| | PCNA, Mean Density/μg | BrdU |
|---|---|---|
| Groups | protein | Labeling Index, % |
| Ungrafted vein | 0 (n = 6) | ND |
| Vehicle | 24.13 ± 3.68 (n = 6) | 25.1 ± 1.7 (n = 3) |
| E2F decoy ODN | 0.79 ± 0.79*^{x} (n = 6) | 8.3 ± 2.8*^{x} (n = 3) |
| Scrambled ODN | 17.87 ± 4.76 (n = 6) | 24.9 ± 2.3 (n = 3) |

PCNA indicates proliferating cell nuclear antigen; BrdU, Bromodeoxyuridine; and ODN, oligonucleotides. Values are mean±SEM. P<0.05 for E2F decoy ODN vs vehicle* and scrambled ODN^{x} groups. ND indicates not determined.

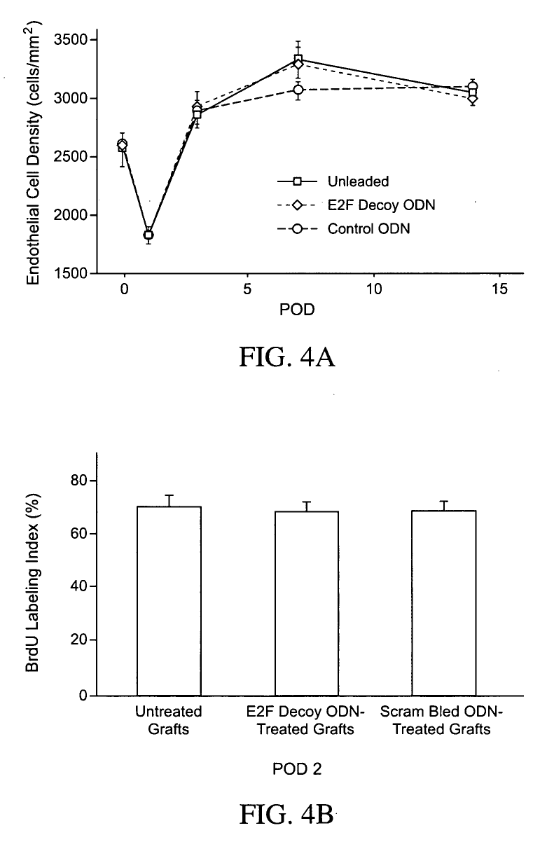

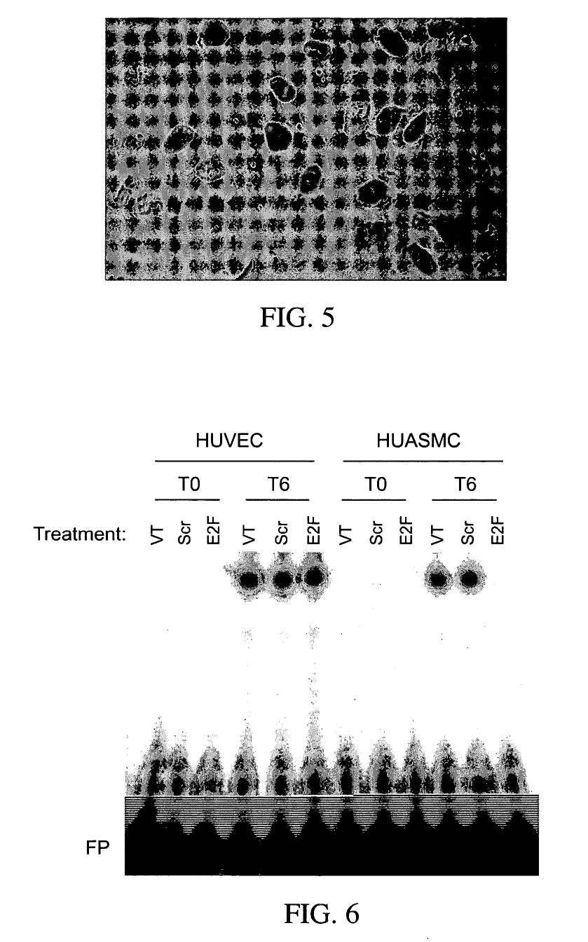

Given the dependence of endothelial healing on the burst of proliferative activity in the early postoperative period, we examined endothelial healing in grafts once again treated with either E2F decoy or a control scrambled sequence ODN. The increase in cell density observed in vehicle-treated grafts at each time point was essentially unchanged in vehicle and scrambled ODN-treated as well as E2F decoy ODN-treated vessels (P<0.4) (FIG. 4A). This observation was further confirmed with BrdU labeling of endothelial monolayers with the Häutchen technique (FIG. 4B). To document the successful delivery of ODN into the endothelial cells, the transfection of grafts with biotinylated ODN was examined. Our result confirmed nuclear localization of labeled ODN in >75% of cells by streptavividin-peroxidase staining of isolated vein graft endothelial monolayer (FIG. 5).

Response of Endothelial Cells and VSMC In Vitro to E2F Decoy ODN Treatment

Having demonstrated a differential response to E2F decoy ODN treatment, we sought to test the hypothesis that normal endothelial healing in ODN-treated grafts reflects an ability of endothelial cells to mount a proliferative response despite the presence of E2F decoy ODN in a cell culture system. Despite a similar degree of nuclear localization of FITC labeled ODN in the HUASMC and HUVEC cultures (70 to 80% in both), HUASMC proliferation was inhibited by E2F decoy ODN treatment (41.7±4.4% inhibition versus vehicle treated control, P<0.001), whereas this treatment did not affect the proliferation of HUVEC (1.9±5.6%, P<0.5) (n=6 per treatment group). By using the electrophoretic mobility shift assay, it was possible to confirm an effective reduction in E2F binding activity after serum stimulation in HUASMC treated with E2F decoy that was not seen in scrambled ODN-treated cells (FIG. 6). In HUVEC, however, there was no significant reduction of serum-induced E2F binding activity after E2F decoy treatment. Supershift with anti-E2F-1 monoclonal antibody and competition with unlabeled probe confirmed the predominant role of this E2F isoform and the shown to redirect vein graft biology away from neointimal hyperplasia and toward medial hypertrophy as an adaptive response to the hemodynamic stress of the arterial circulation. The integrity of the endothelium is believed to play an important role in the prevention of vascular proliferative diseases such as atherosclerosis, which is responsible for the majority of bypass vein graft failures (Cox et al., Prog. Cardiovasc. Disc. 34:45-68 (1991)). Despite traumatic and biological injury to the endothelium during vein grafting, including acute stretch, several groups have documented an intact vein graft endothelium by postoperative day 7 (Zwolak et al. J. Vasc. Surg. 5:126-136 (1987); Davies et al., Ann. Vasc. Surg. 13:378-385 (1999)). In this study, we sought to characterize this endothelial cell healing response during the first week after experimental vein grafting and measure the effect of treatment with E2F decoy ODN.

We observed a rapid, synchronized burst of endothelial proliferative activity in response to acute stretching of the native vein graft wall. This response was documented both by electron microscope analysis of cell density and cell number as well as immunohistochemical analysis of BrdU incorporation. This rapid proliferative response was uniformly distributed throughout the length of the vein graft and occurred despite the apparent preservation of cell-cell contact on electron microscopic analysis of silver-stained tissue specimens. Previous studies have begun to examine the morphological and biological responses of endothelial cells to physical strain on the cell membrane. Cyclic stretch of endothelial cells in culture, as well as other cell membrane perturbations, has been associated with cell cycle stimulation and a rapid increase in adenylate cyclase activation (Davies, Flow Dependent Regulation of Vascular Function in Health and Disease, New York, N.Y.; Academic Press 1984, 46-61; DePaola et al., Arterioscler. Throm. 12:1254-1257 (1992)). In this study, the decline of endothelial cell proliferation back to baseline occurred within a time frame of several days and was associated with the restoration of normal endothelial cell density.

Surprisingly, this rapid and synchronized endothelial proliferative response in the graft was not hindered by efficient nuclear delivery of E2F decoy ODN, despite the simultaneous blockade of cell cycle progression in VSMC within the same vessel. This differential response of endothelial cells and VSMC was further confirmed in studies of cultured cells that revealed an endothelial cell resistance to E2F decoy-mediated cell cycle arrest. We speculate that the properties that lead to this differential response, which has also been suggested by previous studies involving antiproliferative ODN treatment of denuded arteries, (Bennett et al., Arterioscler. Thromb. Vasc. Biol. 17:2326-2332 (1997)) may include differences in ODN metabolism or in the ability of endothelial cells to respond to ODN-mediated gene blockade by further augmenting upregulated expression of cell cycle. regulatory proteins. Additionally, cyclin E-driven pathways of cell cycle activation have been described that are relatively independent of E2F activity; under certain conditions, cells have been shown to progress through S, G2, and M phases in the absence of E2F transactivation (Lukas et al., Genes Dev. 11:1479-1492 (1997)). It is not known how common this type of cyclin E-dependent cell cycle progression may be in different cell types. As demonstrated in this study, however, endothelial cells appear to be programmed to support a burst of proliferative activity that may at times be critical for maintenance of physiological homeostasis. Such an epithelial population may have multiple alternative pathways of cell cycle regulation to ensure its capacity for a brief, rapid proliferative response that are not available to smooth muscle cells. Finally, there are numerous members of the E2F family of transcription factors that play differing stimulatory and inhibitory roles in cell cycle regulation (Glaubatz et al., Proc. Natl. Acad. Sci. USA 95:9190-9195 (1998)). Differential expression of these factors in different cell types might result in a varied response to decoy treatments. The profiles of E2F family member expression in endothelial cells and VSMC have not yet been characterized, and further investigation into these profiles, along with an analysis of the decoy's affinity for different family members, may shed further light onto the mechanism of this differential response to the ODN therapy. In any event, E2F decoy ODN treatment of vascular grafts inhibits VSMC proliferation and activation but spares the endothelium, thereby allowing normal endothelial healing. The improved endothelial cell function observed in previous studies at 4 to 6 weeks after operation probably is related to the inhibition of neointimal hyperplasia and to a subsequent decrease in local concentrations of cytokines and other proinflammatory molecules that are released by activated VSMC and leukocytes within the neointimal layer. The differential responses of endothelial cells and VSMC to E2F decoy observed in this study may have important implications for the therapeutic blockade of cell cycle progression in treating postangioplasty restenosis, native arterial atherosclerosis, and transplantation vasculopathy.

### EXAMPLE 2

Gel Shift for Human Aortic Smooth Muscle Cells

Coronary Artery Endothelial (Clonetics; HCAEC; CC-2585), Smooth Muscle Cells (Clonetics; CASMC; CC-2583), and a human aortic smooth muscle cell line (T/G HA-VSMC) was grown to approximately 80% confluence and serum-starved (in F12K media+0.5% BSA) for 48 hours to render the cells quiescent. The cells were then serum stimulated (in complete regular growth media but with 20% FBS) for 22 hours. Nuclear extracts were prepared according to the standard protocol of Dignam et al and the protein concentration was determined.

A double-stranded oligonucleotide containing the binding site for E2F (^{5′}CTAGATTTCCCGCGGATC^{3′}) (SEQ ID NO: 5) was end-labeled with [γ-³²P] ATP and T4 Polynucleotide Kinase. Equal amounts of nuclear extract (8 μg) were incubated with the radiolabeled probe in a reaction solution containing 0.5 μg poly dIdC and a reaction binding buffer (10 mM Tris-HCl pH 7.4, 40 mM KCl, 1 mM EDTA, 1 mM DTT, 0.05% NP-40, 8% glycerol) in a total reaction volume of 20 μl. To determine the identity of the bands, some reactions also contained 4 ug of antibody. After a 30 minute incubation at room temperature, the reactions were loaded on a non-denaturing polyacrylamide gel, dried, exposed to a phosphor screen and scanned on a Typhoon 8600 Phosphor Imager (Amersham).

The antibodies used in this experiment were all purchased from Santa Cruz Biotechnology and were as follows: E2F-1 (sc-251X (KH95), specific for mouse, rat, human E2F-1, mouse monoclonal); E2F-2 (sc-632X (L-20), specific for mouse, rat, human E2F-2, affinity-purified rabbit polyclonal, not cross-reactive to E2F-1, E2F-3, E2F-4 or E2F-5); E3 (sc-878X (C-18), specific for mouse, rat, human E2F-3, affinity-purified rabbit polyclonal); E2F-4 (sc-866X (C-20), specific for mouse, rat, human E2F-4, affinity-purified rabbit polyclonal, not cross-reactive to E2F-1, E2F-2, E2F-3 or E2F-5).

The supershift experiments comparing the E2F complexes bound by E2F Decoy revealed different complexes in endothelial cells compared to smooth muscle cells. These differences reveal that there are differences in either the identity of E2F complexes or the affinity of different complexes for E2F Decoy in endothelial cells compared to smooth muscle cells. The differences in binding of E2F Deocy to E2F complexes in smooth muscle cells vs. endothelial cells may lead to differential activity of E2F Decoy in these cell types.

All references cited throughout the disclosure are hereby expressly incorporated by reference. Although the invention is illustrated by reference to certain embodiments, it is not so limited. One skilled in the art will appreciate that further modifications and variations are possible, without diverting from the basic idea of the present invention. All such modifications and variations are specifically intended to be within the scope herein.

What is claimed is:

1. A method for selective inhibition of vascular smooth muscle cell proliferation comprising: (a) delivering to a blood vessel in need of endothelial healing an E2F decoy oligonucleotide, and (b) determining that vascular smooth muscle cell proliferation is inhibited without substantial inhibition of endothelial cell proliferation or function.

2. The method of claim 1 wherein the blood vessel is a vein.

3. The method of claim 2 wherein the blood vessel is a vein graft.

4. The method of claim 1 wherein the blood vessel is an artery.

5. The method of claim 1 wherein the E2F oligonucleotide decoy is delivered by pressure-mediated transfer.

6. The method of claim 1 wherein the E2F decoy oligonucleotide is delivered ex vivo.

7. The method of claim 1 wherein step (b) comprises monitoring endothelial healing following delivery of the E2F decoy oligonucleotide.

8. The method of claim 1 wherein the E2F decoy oligonucleotide is delivered to a blood vessel of a mammal following vascular trauma.

9. The method of claim 8 wherein the mammal is a human.

10. The method of claim 9 wherein the vascular trauma is due to a surgical procedure.

11. The method of claim 9 wherein the vascular trauma is due to vein or artery grafting or angioplasty.

12. The method of claim 11 wherein the E2F decoy oligonucleotide is delivered within one week following vein or artery grafting or angioplasty.

13. The method of claim 11 wherein the E2F decoy oligonucleotide is delivered during or immediately following vein or artery grafting or angioplasty.

14. The method of claim 11 wherein the E2F decoy oligonucleotide is delivered by coating or impregnating an implantable device.

15. The method of claim 14 wherein the implantable device is a cardiac stent.

16. The method of claim 14 wherein the implantable device is a renal stent.

17. The method of claim 16 wherein the implantable device is a renal artery stent.

18. The method of claim 14 wherein the implantable device is an artificial conduit.

19. A method for prevention of stenosis or restenosis, comprising (a) delivering to a blood vessel at risk of stenosis or restenosis an E2F decoy oligonucleotide, and (b) determining that vascular smooth muscle cell proliferation is inhibited without substantial inhibition of endothelial cell proliferation or function.

20. A method for the treatment of stenosis or restenosis, comprising (a) delivering to a blood vessel exhibiting signs of stenosis or restenosis an E2F oligonucleotide, and (b) determining that vascular smooth muscle cell proliferation is inhibited without substantial inhibition of endothelial cell proliferation or function.
